(19) United States
(12) Patent Application Publication
(10) Pub. No.: US 2006/0167076 A1
(43) Pub. Date: Jul. 27, 2006

(54) Acutumine and acutumine compounds synthesis and use

(76) Inventors: Guo-Wei Qin, Shanghai (CN); Xi-Can Tang, Shanghai (CN); Pierre Lestage, La Celle Saint Cloud (FR); Daniel-Henri Caignard, Le Pecg (FR); Pierre Renard, Le Chesnay (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/519,418

(22) PCT Filed: Jun. 16, 2003
(86) PCT No.: PCT/IB03/02600

§ 371 Date: Dec. 22, 2004

(30) Foreign Application Priority Data
Jun. 25, 2002 (CN) 02121479.4

(51) Int. Cl. A61K 31/403; C07D 209/96

(52) U.S. Cl. 514/409; 548/407

(57) ABSTRACT

The invention relates to acutumine and compounds thereof and also to compounds of formula (I): 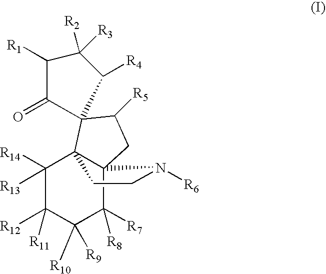 wherein
R₁ and R₂ each represent hydrogen or together form an additional bond,
R₃ represents hydrogen or alkoxy,
R₅ represents hydrogen or chlorine,
R₆ represents hydrogen or alkyl, alkylcarbonyl or aroyl,
R₇ and R₁₀ each represent alkoxy,
R₁₀ represents alkoxy,
R₄, R₈, R₉, R₁₁, R₁₂, R₁₃ and R₁₄ are as defined in the description. and medicinal products containing the same which are useful in treating deficiencies of memory.

Menispermum dauricum is a ligneous climbing plant, more than ten metres long, which is widespread in the North, North-East and East of China (Editorial Board, “National Collective Data of Chinese Traditional and Herbal Medicines”, Peoples Health Publisher, First Edition (Chinese), 1975, p. 105). The dry rhizome, designated Rhizoma Menispermi, is part of traditional Chinese medicine and is now officially included in the Chinese Pharmacopoeia as an analgesic and antipyretic (Pharmacopoeia Committee of People's Republic of China, 2000).

The active principles present in Menispermum dauricum are essentially alkaloids (1 to 2% of the crude extract). Numerous alkaloids having various structures such as bisbenzylisoquinoline, oxoisoaporphine, aporphine, proaporphine, morphinan and many others have been isolated and characterised.

A number of alkaloids have been purified and studied for their pharmacological properties. For example, dauricine, a major alkaloid constituent of the rhizome, has been found to be active in the cardiovascular system and has anti-inflammatory properties. It has been used clinically for treating arrhythmia patients.

Dahurisoline, another alkaloid having a bisbenzylisoquinoline structure, has exhibited muscle-relaxant effects (Liu Chang-Xiao et al., “Modern Research and Application of Chinese Medicinal Plants”, Hong Kong Medical Publisher, First Edition (English) in 2000, p. 480).

Acutumine, a minor alkaloid constituent of the rhizome, was discovered in 1967 and has the special characteristic of containing a chlorine atom (Tomita, M. et al., Chemical and Pharmaceutical Bulletin, 1971, 19(4), p. 770). We have now discovered that acutumine has mnemocognition-facilitating properties in animal experimental models.

Ageing of the population due to increased life expectancy has brought with it a major increase in cognitive disorders associated with normal cerebral ageing or pathological cerebral ageing occurring in the course of neurodegenerative diseases such as, for example, Alzheimer's disease.

The majority of substances used today in treating cognitive disorders associated with ageing act by facilitating the central cholinergic systems—either directly, as in the case of acetylcholinesterase inhibitors (tacrine, donepezil) and cholinergic agonists (nefiracetam), or indirectly, as in the case of nootropic agents (piracetam, pramiracetam) and cerebral vasodilators (vinpocetine).

It has been therefore been especially valuable to synthesise new compounds that are capable of opposing the cognitive disorders associated with ageing and/or of improving cognitive processes.

The present invention relates, on the one hand, to the use of acutumine 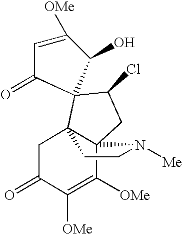
and/or acutumine compounds in mnemocognitive disorders and, on the other hand, to the synthesis of new compounds having especially valuable pharmacological properties in the same area.

The present invention relates more specifically to compounds of formula (I): 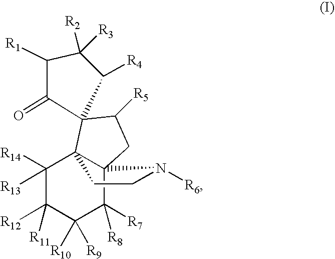
wherein
R₁ and R₂ each represent a hydrogen atom or together form an additional bond,
R₃ represents a hydrogen atom or an alkoxy group,
R₄ represents a hydrogen atom or a hydroxy, alkoxy, alkylcarbonyloxy or arylcarbonyl-oxy group,
R₅ represents a hydrogen or halogen atom,
R₆ represents a hydrogen atom or an alkyl, alkylcarbonyl or aroyl group,
R₇ represents an alkoxy group,
R₈ and R₉ together form an additional bond,
   or R₈ and R₁₃ together form a sulphide bridge and, in that case, R₉ and R₁₀ together form an oxo group and R₁₄ represents a chlorine atom,
R₁₀ represents an alkoxy group,
R₁₁ represents a hydroxy or alkoxy group,
R₁₂ represents a hydrogen atom,
or R₁₁ and R₁₂ together form an oxo, oxime or O-alkyl-oxime group,
and R₁₃ and R₁₄ each represent a hydrogen atom or together form an oxo group,
   with the proviso that the compound of formula (I) cannot represent:
spiro[(4S,S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one](acutumine)
spiro[(4S,5S)-4-acetyl-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-acetyl-3-methoxy-2-cyclopenten-1-one-5:3(2S)-2-chloro-3aS,7aS-((2,3)-1-acetylpyrrolidine)-6,7-dimethoxy-1,2,3,3a,4,7a-hexahydro-5H-inden-5-one]
spiro[(4S,5S)-4-(benzoyloxy)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-hydroxy-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-ol]
spiro[(4S,5S)-4-hydroxy-3-methoxy-2-cyclopenten-1-one-5:3(2S)-3aS,7aS-((2,3)-1-methylpyrrolidine)-6,7-dimethoxy-1,2,3,3 a,4,7a-hexahydro-5H-inden-5-one]
spiro[(4R,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-(benzoyloxy)-3-methoxy-2-cyclopenten-1-one-5:3(2S)-2-chloro-3aS,7aS-((2,3)-1-benzoylpyrrolidine)-6,7-dimethoxy-1,2,3,3a,4,7a-hexahydro-5H-inden-5-one]
spiro[(4S,5S)-4-acetyl-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[4S,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one](acutumidine)
spiro[4R,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one]
spiro[(5S)-2-methoxy-2-cyclopenten-1-one-5:3-3aS,7aS-((2,3)-1H-pyrrolidine)-6,7-dimethoxy-1,2,3,3a,4,7a-hexahydro-5H-inden-5-one]
spiro[(5S)-2-methoxy-2-cyclopenten-1-one-5:3-2-chloro-3aS,7aS-((2,3)-1H-pyrrolidine)-6,7-dimethoxy-1,2,3,3a,4,7a-hexahydro-5H-inden-5-one],
   it being understood that
“alkyl” means an alkyl group containing 1 to 6 carbon atoms which may be linear or branched,
“alkoxy” means an alkyloxy group containing 1 to 6 carbon atoms which may be linear or branched,
“aryloxy” means an aryloxy group wherein the aryl moiety represents a phenyl or naphthyl group,
“aroyl” means an arylcarbonyl group wherein the aryl moiety represents a phenyl or naphthyl group,
   to their enantiomers and diastereoisomers, and to addition salts thereof with a pharmaceutically acceptable acid or base.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, oxalic acid, etc.

Among the pharmaceutically acceptable bases there may be mentioned, without implying any limitation, sodium hydroxide, potassium hydroxide, triethylamine, tert-butylamine etc.

The preferred configuration of compounds of formula (I) according to the invention is that shown in formula (I′):

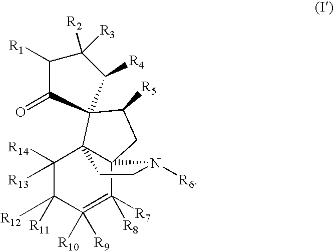

Preferred compounds of the invention are compounds of formula (I) wherein R₁ and R₂, on the one hand, and R₈ and R₉, on the other hand, together form an additional bond.

The preferred meaning of groups R₃, R₇ and R₁₀ of compounds of formula (I) according to the invention is the methoxy group.

Advantageously, R₄ represents a hydroxy, acetyloxy or benzoyloxy group.

Very preferably, R₅ represents a chlorine atom.

R₆ more especially represents a methyl or ethyl group or a hydrogen atom.

The invention preferably relates to compounds of formula (I) wherein R₁₁ and R₁₂ together form an oxo group.

More especially, R₁₃ and R₁₄ each represent a hydrogen atom.

Even more advantageously, the invention relates to compounds of formula (I) which are:
spiro[(4S,5S)-4-(ethoxycarbonyl)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-hydroxy-2-cyclopenten-1-one-5:3(2S)-2-chloro-3aS,7aS-((2,3)-1-ethylpyrrolidine)-6,7-dimethoxy-1,2,3,3a,4,7a-hexahydro-5H-inden-5-one]
spiro[(4S,5S)-4-(ethoxycarbonyl)-3-methoxy-2-cyclopenten-1-one-5:3(2S)-2-chloro-3aS,7aS-((2,3)-1-propanoylpyrrolidine)-6,7-dimethoxy-1,2,3,3a,4,7a-hexahydro-5H-inden-5-one]
spiro[(4S,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one oxime]
spiro[(4S,5S)-3,4-dimethoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4R,5S)-4-hydroxy-3-methoxy-2-cyclopenten-1-one-5:3(2S)-2-chloro-3aS,7aS-((2,3)-1-methylpyrrolidine)-6,7-dimethoxy-2,3,3a,7a-tetrahydro-4H,5H-indene-4,5-dione]
spiro[(5S)-3-methoxy-2-cyclopenten-1-one-5:3(2S)-2-chloro-3aS,7aS-((2,3)-1-methyl-pyrrolidine)-6,7-dimethoxy-1,2,3,3a,4,7a-hexahydro-5H-inden-5-one]
spiro[(4S,5S)-4-hydroxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-ol]
spiro[(4R,5S)-4-hydroxy-3-methoxy-2-cyclopenten-1-one-5:3(2S)-2,4-dichloro-3aS,7aS-((2,3)-1-methylpyrrolidine)-7-methoxy-8-thiabicyclo[2.2.1]-1,2,3,3a,4,7a-hexahydro-5H,6H-indene-5,6-dione].

The enantiomers and diastereoisomers and addition salts with a pharmaceutically acceptable acid or base of the preferred compounds of the invention form an integral part of the invention.

The invention relates also to a process for the preparation of compounds of formula (I), which process is characterised in that there is used as starting material the compound of formula (II) (acutumidine): 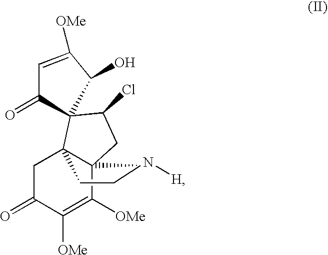
which is subjected to the action of, successively, a demethylating agent and then an alkylating agent to obtain the compound of formula (I/a), a particular case of the compounds of formula (I): 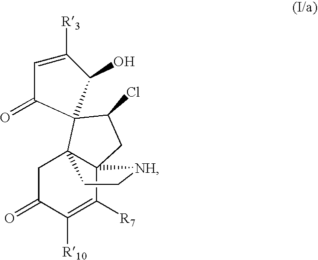
wherein R′₃ and R′₁₀ each represent an alkoxy group and R₇ is as defined for formula (I), which may be subjected to the action of a compound of formula R₁₅CHO (wherein R₁₅ represents an alkyl group) in a reducing medium to obtain the compound of formula (I/b), a particular case of the compounds of formula (I): 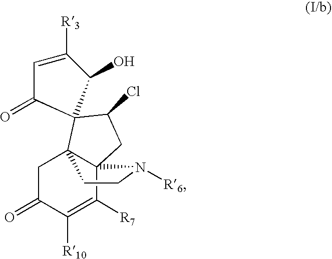
wherein R′₃, R₇ and R′₁₀ are as defined hereinbefore and R′₆ represents an alkyl group,

which compounds of formula (II), (I/a) or (I/b) are subjected to the action of a compound of formula (R₁₆CO)₂O (wherein R₁₆ represents an alkyl or aryl group) to yield the compound of formula (I/c), a particular case of the compounds of formula (I): 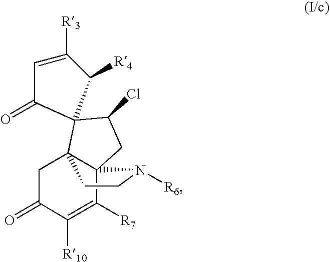
wherein R′₃, R₇ and R′₁₀ are as defined hereinbefore, R₆ is as defined for formula (I) and R′₄ represents a hydroxy, alkylcarbonyloxy or arylcarbonyloxy group,

or which compounds of formula (II), (I/a), (I/b) or (I/c) may be subjected to the action of a compound of formula E-R₁₅ (wherein R₁₅ represents an alkyl group and E represents a leaving group such as a halogen atom or a tosyl group) to yield the compound of formula (I/d), a particular case of the compounds of formula (I): 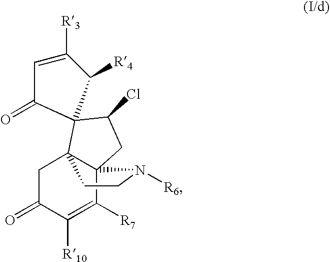
wherein R′₃, R₆, R₇ and R′₁₀ are as defined hereinbefore and R₄ is as defined for formula (I),

which may be subjected to the action of the compound of formula R₁₇ONH₂ wherein R₁₇ represents a hydrogen atom or an alkyl group to yield the compound of formula (I/e), a particular case of the compounds of formula (I): 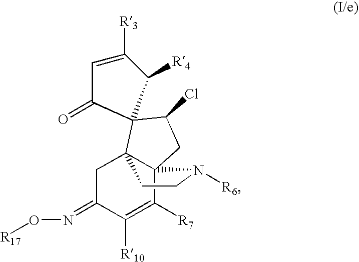
wherein R′₃, R₄, R₆, R₇, R′₁₀ and R₁₇ are as defined hereinbefore,

or which compound of formula (I/d) may be subjected to the action of SOCl₂/DMF to obtain the compounds of formula (I/f), particular cases of the compounds of formula (I): 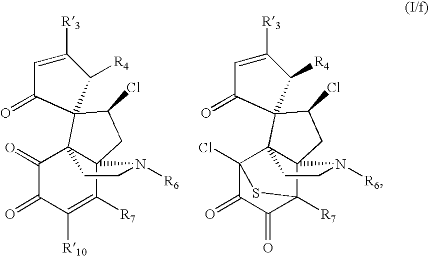
wherein R′₃, R₄, R₆, R₇ and R′₁₀ are as defined hereinbefore,

or which compound of formula (I/d) may be subjected to the action of a reducing agent such as LiAlH₄ to obtain the compounds of formula (I/g), particular cases of the compounds of formula (I): 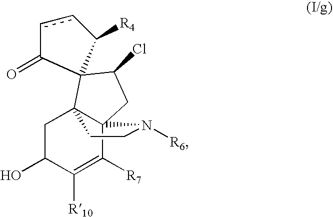
wherein R₄, R₆, R₇ and R′₁₀ are as defined hereinbefore and the symbol [symbol] indicates that the bond may be single or double,

or which compound of formula (I/d), (I/e), (I/f) or (I/g) may be subjected to the action of n-Bu₃SnH in the presence of AIBN to obtain the compounds of formula (I/h), particular cases of the compounds of formula (I): 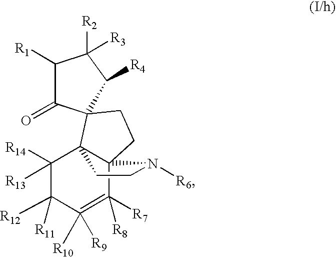
wherein R₄, R₆ and R₇ are as defined hereinbefore and R₁, R₂, R₃, R₅, R₈, R₉, R₁₀, R₁₁, R₁₂, R₁₃ and R₁₄ are as defined for formula (I),

the compounds of formulae (I/a) to (I/h) constituting the totality of the compounds of the invention, which may be purified according to a conventional separation technique, are converted, if desired, into their addition salts with a pharmaceutically acceptable acid or base and are separated, where appropriate, into their isomers according to a conventional separation technique.

The compound of formula (II) can be obtained by the person skilled in the art by means of extraction starting from Menispermum dauricum rhizome according to the procedure of FIG. 1:

[image]

Besides the fact that the compounds of the present invention are new, they possess properties of facilitating cognitive processes, making them of use in the treatment of cognitive deficiencies associated with cerebral ageing and with neurodegenerative diseases such as Alzheimer's disease, Parkinson's disease, Pick's disease, Korsakoff's disease, and frontal lobe and subcortical dementias.

The invention relates also to pharmaceutical compositions comprising as active ingredient at least one compound of formula (I) together with one or more appropriate, inert, non-toxic excipients.

The Applicant has moreover discovered that acutumine and/or acutumine compounds have mnemocognition-facilitating properties.

The invention accordingly relates also to the use of acutumine and/or acutumine compounds in obtaining pharmaceutical compositions for use in the treatment of cognitive deficiencies associated with cerebral ageing and with neurodegenerative diseases such as Alzheimer's disease, Parkinson's disease, Pick's disease, Korsakoff's disease, and frontal lobe and subcortical dementias.

More especially, the invention relates to the use, in obtaining pharmaceutical compositions for use in the treatment of cognitive deficiencies associated with cerebral ageing and with neurodegenerative diseases, of acutumine and/or acutumine compounds such as, for example:
spiro[(4S,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5H-one](acutumine)
spiro[(4S,5S)-4-acetyl-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-acetyl-3-methoxy-2-cyclopenten-1-one-5:3(2S)-2-chloro-3aS,7aS-((2,3)-1-acetylpyrrolidine)-6,7-dimethoxy-1,2,3,3a,4,7a-hexahydro-5H-inden-5-one]
spiro[(4S,5S)-4-(benzoyloxy)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-hydroxy-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-ol]
spiro[(4S,5S)-4-hydroxy-3-methoxy-2-cyclopenten-1-one-5:3(2S)-3aS,7aS-((2,3)-1-methylpyrrolidine)-6,7-dimethoxy-1,2,3,3a,4,7a-hexahydro-5H-inden-5-one]
spiro[(4R,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-(benzoyloxy)-3-methoxy-2-cyclopenten-1-one-5:3(2S)-2-chloro-3aS,7aS-((2,3)-1-benzoylpyrrolidine)-6,7-dimethoxy-1,2,3,3a,4,7a-hexahydro-5H-inden-5-one]
spiro[(4S,5S)-4-acetyl-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[4S,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one](acutumidine)
spiro[4R,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one]
spiro[(5S)-2-methoxy-2-cyclopenten-1-one-5:3-3aS,7aS-((2,3)-1H-pyrrolidine)-6,7-dimethoxy-1,2,3,3a,4,7a-hexahydro-5H-inden-5-one]
spiro[(5S)-2-methoxy-2-cyclopenten-1-one-5:3-2-chloro-3aS,7aS-((2,3)-1H-pyrrolidine)-6,7-dimethoxy-1,2,3,3a,4,7a-hexahydro-5H-inden-5-one].

An advantageous aspect of the invention relates to the use of acutumine in obtaining pharmaceutical compositions for use in the treatment of cognitive deficiencies associated with cerebral ageing and with neurodegenerative diseases.

Another especially interesting aspect of the invention relates to the use, in obtaining pharmaceutical compositions for use in the treatment of cognitive deficiencies associated with cerebral ageing and with neurodegenerative diseases, of spiro[(4S,5S)-4-hydroxy-3-methoxy-2-cyclopenten-1-one-5:3(2S)-2-chloro-3aS,7aS-((2,3)-1-methyl-pyrrolidine)-6,7-dimethoxy-1,2,3,3a,4,7a-hexahydro-5H-inden-5-one], of spiro[(4S,5S)-4-acetyl-3-methoxy-2-cyclopenten-1-one-5:3(2S)-2-chloro-3aS,7aS-((2,3)-1-methyl-pyrrolidine)-6,7-dimethoxy-1,2,3,3a,4,7a-hexahydro-5H-inden-5-one], of spiro[(4S,5S)-4-acetyl-3-methoxy-2-cyclopenten-1-one-5:3(2S)-2-chloro-3aS,7aS-((2,3)-1-acetylpyrrolidine)-6,7-dimethoxy-1,2,3,3a,4,7a-hexahydro-5H-inden-5-one], of spiro[(4S,5S)-4-(benzoyloxy)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one], of spiro[(4S,5S)-4-hydroxy-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-ol], of spiro[(4S,5S)-4-hydroxy-3-methoxy-2-cyclopenten-1-one-5:3(2S)-3aS,7aS-((2,3)-1-methylpyrrolidine)-6,7-dimethoxy-1,2,3,3a,4,7a-hexahydro-5H-inden-5-one], of spiro[(4R,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one], of spiro[(4S,5S)-4-(benzoyloxy)-3-methoxy-2-cyclopenten-1-one-5:3(2S)-2-chloro-3aS,7aS-((2,3)-1-benzoylpyrrolidine)-6,7-dimethoxy-1,2,3,3a,4,7a-hexahydro-5H-inden-5-one], of spiro[(4S,5S)-4-acetyl-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-one), of spiro[4S,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one](acutumidine), of spiro[4R,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one], of spiro[(5S)-2-methoxy-2-cyclopenten-1-one-5:3-3aS,7aS-((2,3)-1H-pyrrolidine)-6,7-dimethoxy-1,2,3,3a,4,7a-hexahydro-5H-inden-5-one]and of spiro[(5S)-2-methoxy-2-cyclopenten-1-one-5:3-2-chloro-3aS,7aS-((2,3)-1H-pyrrolidine)-6,7-dimethoxy-1,2,3,3a,4,7a-hexahydro-5H-inden-5-one].

The invention relates also to pharmaceutical compositions comprising acutumine or a compound thereof, in combination with one or more pharmaceutically acceptable excipients, for use in the treatment of cognitive deficiencies associated with cerebral ageing and with neurodegenerative diseases such as Alzheimer's disease, Parkinson's disease, Pick's disease, Korsakoff's disease, and frontal lobe and subcortical dementias.

Among the pharmaceutical compositions according to the invention, there may be mentioned more especially those that are suitable for oral, parenteral (intravenous or subcutaneous) or nasal administration, tablets or dragées, sublingual tablets, gelatin capsules, lozenges, suppositories, creams, ointments, dermal gels, injectable preparations, drinkable suspensions etc.

The useful dosage can be varied according to the nature and severity of the disorder, the administration route and also the age and weight of the patient. The dosage varies from 0.01 mg to 1 g per day in one or more administrations.

The following Examples illustrate the invention but do not limit it in any way.

### EXAMPLE 1

### Spiro[(4S,5S)-4-(ethoxycarbonyl)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]

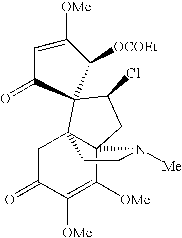

### Step A: Spiro[(4S,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]

One gram of the compound of formula (II) is dissolved in HCOOH (10 ml) and stirred with 10 ml of formic aldehyde at 40-50° C. for 4 hours. The reaction mixture is then made alkaline using NH₄OH until a pH of 8-9 is obtained. The white precipitate formed is filtered off and is then dried with K₂CO₃ to yield the title compound.

### Step B: Spiro[(4S,5S)-4-(ethoxycarbonyl)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]

One gram of the compound obtained in Step A is dissolved in CHCl₃and DMF. 2 ml of propanoic anhydride are then added dropwise and the reaction mixture is stirred overnight. Saturated NaHCO₃ solution is then added until a pH of 8-9 is obtained, and the reaction mixture is extracted with CHCl₃. After evaporating off the solvents, the residue obtained is chromatographed on silica gel (CHCl₃:Me₂CO/20:11) to yield the title compound.

Melting Point: 156-158° C.

Elemental Microanalysis:

| | C | H | N |
|---|---|---|---|
| % calculated: | 58.21 | 6.22 | 3.09 |
| % found: | 58.00 | 6.27 | 3.03 |

### EXAMPLE 2

### Spiro[(4S,5S)-4-hydroxy-3-methoxy-2-cyclopenten-1-one-5:3(2S)-2-chloro-3aS,7aS-((2,3)-1-ethylpyrrolidine)-6,7-dimethoxy-1,2,3,3a,4,7a-hexahydro-5H-inden-5-one]

Fifty milligrams of the compound of formula (II) are dissolved in HCOOH (0.5 ml) and stirred with 0.5 ml of acetaldehyde at 40-50° C. for 6 hours. The reaction mixture is then made alkaline using NH₄OH until a pH of 8-9 is obtained and the mixture is extracted with CHCl₃. The residue obtained after evaporating off the solvent is chromatographed on silica gel (CHCl₃:Me₂CO/2:1) to yield the title compound.

Melting Point: 156-158° C.

Elemental Microanalysis:

| | C | H | N |
|---|---|---|---|
| % calculated: | 58.32 | 6.31 | 3.40 |
| % found: | 57.98 | 6.31 | 3.09 |

### EXAMPLE 3

### Spiro[(4S,5S)-4-(ethoxycarbonyl)-3-methoxy-2-cyclopenten-1-one-5:3(2S)-2-chloro-3aS,7aS-((2,3)-1-propanoylpyrrolidine)-6,7-dimethoxy-1,2,3,3a,4,7a-hexahydro-5H-inden-5-one]

One gram of the compound of formula (II) is dissolved in N,N-dimethylaminopyridine and 2 ml of CHCl₃. 2 ml of acetic anhydride are then added dropwise and the reaction mixture is stirred overnight at ambient temperature. Saturated NaHCO₃ solution is then added until a pH of 8-9 is obtained and the reaction mixture is extracted with CHCl₃. After evaporating off the solvents, the residue obtained is chromatographed on silica gel (CHCl₃:Me₂CO/20:11) to yield the title compound.

Melting Point: 166-168° C.

Elemental Microanalysis:

| | C | H | N |
|---|---|---|---|
| % calculated: | 58.12 | 6.09 | 2.82 |
| % found: | 57.55 | 6.03 | 2.72 |

### EXAMPLE 4

### Spiro[(4S,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one oxime]

One gram of the compound obtained in Step A of Example 1 is stirred in 15 ml of ethanol with 1 g of hydroxylamine at 70-80° C. for 4 hours. Saturated NaHCO₃ solution is then added until a pH of 8-9 is obtained and the reaction mixture is extracted with CHCl₃. After evaporating off the solvents, the residue obtained is chromatographed on silica gel (CHCl₃:Me₂CO/3:1) to yield the title compound in the form of a white solid.

Melting Point: 211-213° C.

Elemental Microanalysis:

| | C | H | N |
|---|---|---|---|
| % calculated: | 55.27 | 6.10 | 6.79 |
| % found: | 55.17 | 5.79 | 7.46 |

### EXAMPLE 5

### Spiro[(4S,5S-3,4-dimethoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]

The compound obtained in Step A of Example 1 (200 mg) is dissolved in DMSO and stirred with 100 mg of NaOH and 1 ml of CH₃I at ambient temperature for 20 minutes. The reaction mixture is then diluted with 5 ml of water and then with CHCl₃. After extracting and evaporating off the solvents, the residue obtained is chromatographed on silica gel (CHCl₃:MeOH/20:1) to yield the title compound in the form of white needles.

Melting Point: 165-167° C.

Elemental Microanalysis:

| | C | H | N |
|---|---|---|---|
| % calculated: | 57.32 | 6.36 | 3.40 |
| % found: | 57.18 | 6.38 | 3.86 |

### EXAMPLE 6

### Spiro[(4R,5S)-4-hydroxy-3-methoxy-2-cyclopenten-1-one-5:3(2S)-2-chloro-3aS,7aS-((2,3)-1-methylpyrrolidine)-6,7-dimethoxy-2,3,3a,7a-tetrahydro-4H,5H-indene-4,5-dione]

The compound obtained in Step A of Example 1 (30 mg) is dissolved in SOCl₂ and is stirred with DMF (catalyst) at 85° C. for 30 minutes. The crude reaction mixture is chromatographed on silica gel (CHCl₃:Et₂O/10:1) to yield the title compound.

Melting Point: 152-154° C.

### EXAMPLE 7

### Spiro[(5S)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]

The title compound was isolated by chromatography on silica gel, starting from the ethanolic extract obtained from Menispermum dauricum rhizome.

Melting Point: 174-176° C.

### EXAMPLE 8

### Spiro[(4S,5S)-4-hydroxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-ol]

The compound obtained in Step A of Example 1 (50 mg) is dissolved in THF (15 ml) and is stirred with LiAlH₄ at ambient temperature for 2 hours. The crude reaction mixture is diluted with water, extracted with CHCl₃ and then chromatographed on silica gel to yield the title compound.

### EXAMPLE 9

### Spiro[(4R,5S)-4-hydroxy-3-methoxy-2-cyclopenten-1-one-5:3(2S)-2,4-di-chloro-3aS,7aS-((2,3)-1-methylpyrrolidine)-7-methoxy-8-thiabicyclo[2.2.1]-1,2,3,3a,4,7a-hexahydro-5H,6H-indene-5,6-dione]

The procedure is as in Example 6 (the two compounds (Examples 6 and 9) are formed in the course of the same reaction sequence).

Melting point: 214-216° C.

### PHARMACOLOGICAL STUDY OF COMPOUNDS OF THE INVENTION

### EXAMPLE A

### Acute Toxicity Study

Acute toxicity was evaluated after oral administration to groups each comprising 8 mice (26±2 grams). The animals were observed at regular intervals during the course of the first day, and daily for the two weeks following treatment. The LD₅₀ (dose that causes the death of 50% of the animals) was evaluated and demonstrated the low toxicity of the compounds of the invention.

### EXAMPLE B

### Morris Water Maze Test in the Mouse

The anti-amnesic effects of the compounds of the present invention have been evaluated using the Morris water maze test (Morris et al., Nature, 1986, 319, 774-776) in the mouse and scopolamine as amnesic agent. Kumming strain mice (18-24 g, Shanghai Experimental Animal Centre) of either sex were used. Mice were placed on the water maze (80×50×20 cm) and trained to find the platform. Following the period of one day's habituation, each mouse received 3 daily training sessions for seven days. Mice were trained to a criterion of finding the platform within 20 seconds and with <2 errors of entering a dead-end. Once a mouse met the criterion, training was reduced to one daily session until all mice met the criterion. Trained mice were randomly assigned to sub-groups. Compounds under study were dissolved in distilled water and administered by the oral route 40 minutes before behavioural testing. Scopolamine (5 mg/kg, i.p.) was injected 30 minutes before the test. The number of errors and the time for reaching the platform were recorded. Data were expressed as means±s.e.m. Statistical analysis was performed using ANOVA followed by Duncan's multiple-range test.

Results demonstrate that compounds of the present invention were capable of counteracting in a dose-dependent manner (from 20 to 100 mg/kg) scopolamine-induced memory impairments in the Morris water maze test in the mouse, indicating that such compounds possess anti-amnesic properties.

As example, compound of Example 1, administered at 60 mg/kg p.o. reach the platform within 18 seconds whereas control animals reach it within 43 seconds.

### EXAMPLE C

### Social Recognition in the Wistar Rat

Initially described in 1982 by THOR and HOLLOWAY (J. Comp. Physiol., 1982, 29 1000-1006), the social recognition test has subsequently been proposed by various authors (DANTZER et al., Psychopharmacology, 1987, 91, 363-368; PERIO et al., Psychopharmacology, 1989, 97, 262-268) for studying the mnemocognitive effects of new compounds. The test is based on the natural expression of the olfactory memory of the rat and its natural tendency to forget and allows evaluation of memorisation, by recognition of a young congeneric animal, by an adult rat. A young rat (21 days), taken at random, is placed for 5 minutes in the cage housing an adult rat. With the aid of a video device, the experimenter observes the social recognition behaviour of the adult rat and measures its overall duration. The young rat is then removed from the adult rat's cage and is placed in its own cage until the second introduction. The adult rat is given the compound under test and, after 2 hours, is again brought into the presence (5 minutes) of the young rat. The social recognition behaviour is then observed again and its duration measured. The assessment criterion is the difference (T₂−T₁), expressed in seconds, between the “recognition” times of the 2 encounters.

The results obtained show a difference (T₂−T₁) ranging from (−20) s to (−45) s for doses ranging from 3 to 30 mg/kg, which shows that the compounds of the invention very greatly enhance memorisation.

As example, compound of Example 4 shows a difference (T₂−T₁) ranging of −45 seconds for an administration of 20 mg/kg.

### EXAMPLE D

### Object Recognition in the Wistar Rat

The object recognition test in the Wistar rat was initially developed by ENNACEUR and DELACOUR (Behav. Brain Res., 1988, 31, 47-59). The test is based on the spontaneous exploratory activity of the animal and has the characteristics of episodic memory in humans. This memory test is sensitive to ageing (SCALI et al., Eur. J. Pharmacol., 1997, 325, 173-180) and to cholinergic dysfunctions (BARTOLINI et al., Pharm. Biochem. Behav. 1996, 53(2), 277-283) and is based on the differences in the exploration of 2 objects of fairly similar shape—one familiar, the other new. Prior to the test, the animals are habituated to the environment (an enclosure without an object). In the course of a first session, the rats are placed (3 minutes) in the enclosure, in which there are 2 identical objects. The duration of exploration is measured for each object. In the course of the second session (3 minutes), 24 hours later, 1 of the 2 objects is replaced by a new object. The duration of exploration is measured for each object. The assessment criterion is the difference, Delta, expressed in seconds, between the exploration times for the new object and for the familiar object in the course of the second session. The control animals, previously treated with the carrier by the IP route 30 minutes before each session, explore the familiar object and the new object in an identical manner, which indicates that the object introduced earlier has been forgotten. Animals treated with a compound that facilitates mnemocognition preferentially explore the new object, which indicates that the object introduced earlier has been remembered.

The results obtained show a difference, Delta, ranging from 5 to 10 s, for doses ranging from 3 to 30 mg/kg, which shows that the compounds of the invention greatly enhance memorisation.

As example, compound of Example 4 shows a Delta of 8 seconds for an administration of 10 mg/kg.

### EXAMPLE E

### Pharmaceutical Composition

Formula for the preparation of 1000 tablets
each containing 10 mg of active ingredient:

| | | |
|---|---|---|
| spiro[(4S,5S)-4-hydroxy-3-methoxy-2-cyclopenten-1-one- | 10 | g |
| 5:3(2S)-2-chloro-3aS,7aS-((2,3)-1-methylpyrrolidine)-6,7- | | |
| dimethoxy-1,2,3,3a,4,7a-hexahydro-5H-inden-5-one oxime] | | |
| (Example 4) | | |
| hydroxypropylcellulose | 2 | g |
| wheat starch | 10 | g |
| lactose | 100 | g |
| magnesium stearate | 3 | g |
| talc | 3 | g |

1-18. (canceled)

19. A compound selected from those of formula (I): 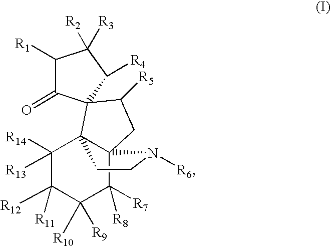
wherein
R₁ and R₂ each represent hydrogen or together form an additional bond,
R₃ represents hydrogen or alkoxy,
R₄ represents hydrogen or hydroxy, alkoxy, alkylcarbonyloxy or arylcarbonyloxy,
R₅ represents hydrogen or halogen,
R₆ represents hydrogen or alkyl, alkylcarbonyl or aroyl,
R₇ represents alkoxy,
R₈ and R₉ together form an additional bond, or R₈ and R₁₃ together form a sulphide bridge and, in that case, R₉ and R₁₀ together form oxo and R₁₄ represents chlorine,
R₁₀ represents alkoxy,
R₁₁ represents hydroxy or alkoxy,
R₁₂ represents hydrogen, or R₁₁ and R₁₂ together form oxo, oxime or O-alkyl-oxime,
and R₁₃ and R₁₄ each represent hydrogen or together form oxo,
with the proviso that the compound of formula (I) cannot represent:
spiro[(4S,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one](acutumine)
spiro[(4S,5S)-4-acetyl-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-acetyl-3-methoxy-2-cyclopenten-1-one-5:3(2S)-2-chloro-3aS,7aS-((2,3)-1-acetylpyrrolidine)-6,7-dimethoxy-1,2,3,3a,4,7a-hexahydro-5H-inden-5-one]
spiro[(4S,5S)-4-(benzoyloxy)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4-hydroxy-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-ol]
spiro[(4S,5S)-4-hydroxy-3-methoxy-2-cyclopenten-1-one-5:3(2S)-3aS,7aS-((2,3)-1-methylpyrrolidine)-6,7-dimethoxy-1,2,3,3a,4,7a-hexahydro-5H-inden-5-one]
spiro[(4R,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[(4S,5S)-4(benzoyloxy)-3-methoxy-2-cyclopenten-1-one-5:3(2S)-2-chloro-3aS,7aS-((2,3)-1benzoylpyrrolidine)-6,7-dimethoxy-1,2,3,3a,4,7a-hexahydro-5H-inden-5-one]
spiro[(4S,5S)-4-acetyl-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-one]
spiro[4S,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one](acutumidine)
spiro[4R,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one]
spiro[(5S)-2-methoxy-2-cyclopenten-1-one-5:3-3aS,7aS-((2,3)-1H-pyrrolidine)-6,7-dimethoxy-1,2,3,3a,4,7a-hexahydro-5H-inden-5-one]
spiro[(5S)-2-methoxy-2-cyclopenten-1-one-5:3-2-chloro-3aS,7aS-((2,3)-1H-pyrrolidine)-6,7-dimethoxy-1,2,3,3a,4,7a-hexahydro-5H-inden-5-one],
it being understood that
“alkyl” means an alkyl group containing 1 to 6 carbon atoms which may be linear or branched,
“alkoxy” means an alkyloxy group containing 1 to 6 carbon atoms which may be linear or branched,
“aryloxy” means an aryloxy group wherein the aryl moiety represents phenyl or naphthyl,
“aroyl” means an arylcarbonyl group wherein the aryl moiety represents phenyl or naphthyl,
its enantiomers and diastereoisomers, and addition salts thereof with a pharmaceutically-acceptable acid or base.

20. A compound according to claim 19, wherein R₁ and R₂, together form an additional bond, and R₈ and R₉ together form an additional bond.

21. A compound according to claim 19, wherein the groups R₃, R₇ and R₁₀ each represent methoxy.

22. A compound according to claim 19, wherein R₄ represents hydroxy, acetyloxy or benzoyloxy.

23. A compound according to claim 19, wherein R₅ represents chlorine.

24. A compound according to claim 19, wherein R₆ represents methyl or ethyl.

25. A compound according to claim 19, wherein R₆ represents hydrogen.

26. A compound according to claim 19, wherein R₁₁ and R₁₂ together form oxo.

27. A compound according to claim 19, wherein R₁₃ and R₁₄ each represent hydrogen.

28. A compound according to claim 19, wherein the compound has the configuration shown by formula (I′):

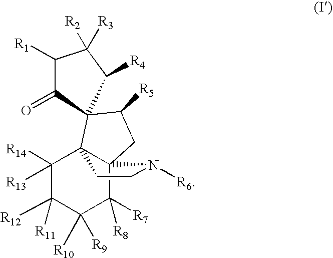

29. A compound according to claim 19, which is selected from
spiro[(4S,5S)-4-(ethoxycarbonyl)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one],
spiro[(4S,5S)-4-hydroxy-3-methoxy-2-cyclopenten-1-one-5:3(2S)-2-chloro-3aS,7aS-((2,3)-1-ethyl-pyrrolidine)-6,7-dimethoxy-1,2,3,3a,4,7a-hexahydro-5H-inden-5-one],
spiro[(4S,5S)-4-(ethoxycarbonyl)-3-methoxy-2-cyclopenten-1-one-5:3(2S)-2-chloro-3aS,7aS-((2,3)-1-propanoylpyrrolidine)-6,7-dimethoxy-1,2,3,3a,4,7a-hexahydro-5H-inden-5-one],
spiro[(4S,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one oxime],
spiro[(4S,5S)-3,4-dimethoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one],
spiro[(4R,5S)-4-hydroxy-3-methoxy-2-cyclopenten-1-one-5:3(2S)-2-chloro-3aS,7aS-((2,3)-1-methylpyrrolidine)-6,7-dimethoxy-2,3,3a,7a-tetrahydro-4H,5H-indene-4,5-dione],
spiro[(5S)-3-methoxy-2-cyclopenten-1-one-5:3(2S)-2-chloro-3aS,7aS-((2,3)-1-methyl-pyrrolidine)-6,7-dimethoxy-1,2,3,3a,4,7a-hexahydro-5H-inden-5-one],
spiro[(4S,5S)-4-hydroxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-ol],
spiro[(4R,5S)-4-hydroxy-3-methoxy-2-cyclopenten-1-one-5:3(2S)-2,4-dichloro-3aS, and
7aS-((2,3)-1-methylpyrrolidine)-7-methoxy-8-thiabicyclo[2.2.1]-1,2,3,3a,4,7a-hexahydro-5H,6H-indene-5,6-dione], its enantiomers, and addition salts thereof with a pharmaceutically acceptable acid or base.

30. A method for treating a living animal body, including a human, afflicted with a condition selected from deficiencies of memory associated with cerebral ageing and with neurodegenerative diseases including Alzheimer's disease, Parkinson's disease, Pick's disease, Korsakoff's disease, and frontal lobe and subcortical dementias, comprising the step of administering to the living animal body, including a human, an amount of a compound of claim 19 which is effective for alleviation of the condition.

31. A pharmaceutical composition useful for the treatment of deficiencies of memory associated with cerebral ageing and with neurodegenerative diseases such as Alzheimer's disease, Parkinson's disease, Pick's disease, Korsakoff's disease, and frontal lobe and subcortical dementias comprising as active principle an effective amount of a compound of claim 19 together with one or more pharmaceutically-acceptable excipients or vehicles.

32. A method for treating a living animal body, including a human, afflicted with a condition selected from deficiencies of memory associated with cerebral ageing and with neurodegenerative diseases including Alzheimer's disease, Parkinson's disease, Pick's disease, Korsakoff's disease, and frontal lobe and subcortical dementias, comprising the step of administering to the living animal body, including a human, an amount of acutumine and/or an acutumine compound which is effective for alleviation of the condition.

33. A method according to claim 32, comprising the step of administering to the living animal body, including a human, an amount of acutumine which is effective for alleviation of the condition.

34. A method according to claim 32, comprising the step of administering to the living animal body, including a human, an amount of an acutumine compound which is effective for alleviation of the condition.

35. A method according to claim 32, wherein the acutumine compound is selected from:
spiro[(4S,5S)-4-hydroxy-3-methoxy-2-cyclopenten-1-one-5:3(2S)-2-chloro-3aS,7aS-(2,3)-1-methylpyrrolidine)-6,7-dimethoxy-1,2,3,3a,4,7a-hexahydro-5H-inden-5-one](acutumine),
spiro[(4S,5S)-4-acetyl-3-methoxy-2-cyclopenten-1-one-5:3(2S)-2-chloro-3aS,7aS-(2,3)-1-methylpyrrolidine)-6,7-dimethoxy-1,2,3, 3a,4,7a-hexahydro-5H-inden-5-one],
spiro[(4S,5S)-4-acetyl-3-methoxy-2-cyclopenten-1-one-5:3(2S)-2-chloro-3aS,7aS-((2,3)-1-acetylpyrrolidine)-6,7-dimethoxy-1,2,3,3a,4,7a-hexahydro-5H-inden-5-one],
spiro[(4S,5S)-4-(benzoyloxy)-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one],
spiro[(4S,5S)-4-hydroxy-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-ol],
spiro[(4S,5S)-4-hydroxy-3-methoxy-2-cyclopenten-1-one-5:3(2S)-3aS,7aS-((2,3)-1-methylpyrrolidine)-6,7-dimethoxy-1,2,3,3a,4,7a-hexahydro-5H-inden-5-one],
spiro[(4R,5S)-4-hydroxy-3-methoxy-2-cyclopenten-1-one-5:3(2S)-2-chloro-3aS,7aS-((2,3)-1-methylpyrrolidine)-6,7-dimethoxy-1,2,3,3a,4,7a-hexahydro-5H-inden-5-one],
spiro[(4S,5S)-4(benzoyloxy)-3-methoxy-2-cyclopenten-1-one-5:3(2S)-2-chloro-3aS,7aS-((2,3)-1benzoylpyrrolidine)-6,7-dimethoxy-1,2,3,3a,4,7a-hexahydro-5H-inden-5-one],
spiro[(4S,5S)-4-acetyl-cyclopentan-1-one-5:3(2S)-2-chloro-3aS,7aS-((2,3)-1-methylpyrrolidine)-6,7-dimethoxy-1,2,3,3a,4,7a-hexahydro-5H-inden-5-one], spiro[4S,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one](acutumidine),
spiro[4R,5S)-4-hydroxy-3-methoxy-2-cyclopenten-1-one-5:3(2S)-2-chloro-3aS,7aS-((2,3)-1H-pyrrolidine)-6,7-dimethoxy-1,2,3,3a,4,7a-hexahydro-5H-inden-5-one],
spiro[(5S)-2-methoxy-2-cyclopenten-1-one-5:3-3aS,7aS-((2,3)-1H-pyrrolidine)-6,7-dimethoxy-1,2,3,3a,4,7a-hexahydro-5H-inden-5-one], and
spiro[(5S)-2-methoxy-2-cyclopenten-1-one-5:3-2-chloro-3aS,7aS-((2,3)-1H-pyrrolidine)-6,7-dimethoxy-1,2,3,3a,4,7a-hexahydro-5H-inden-5-one].

36. A pharmaceutical composition useful for the treatment of deficiencies of memory associated with cerebral ageing and with neurodegenerative diseases such as Alzheimer's disease, Parkinson's disease, Pick's disease, Korsakoff's disease, and frontal lobe and subcortical dementias comprising as active principle, an effective amount of acutumine or an acutumine compound, in combination with one or more pharmaceutically-acceptable excipients or vehicles.
